(19) United States
(12) Patent Application Publication
(10) Pub. No.: US 2010/0063491 A1
(43) Pub. Date: Mar. 11, 2010

(54) DEVICE AND METHOD FOR IMAGING SKIN OBJECTS, AND A METHOD AND DEVICE FOR REDUCING HAIR GROWTH BY MEANS THEREOF

(75) Inventors: Rieko Verhagen, Eindhoven (NL); Bart Willem Jan Spikker, Eindhoven (NL); Robbert Adrianus Maria Van Hal, Eindhoven (NL); Aleksey Kharin, Eindhoven (NL); Natallia Uzunbajakava, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/518,154

(22) PCT Filed: Dec. 7, 2007
(86) PCT No.: PCT/IB07/54969

§ 371 Date: Jun. 8, 2009

(30) Foreign Application Priority Data
Dec. 12, 2006 (EP) 06125915.6

(51) Int. Cl. A61B 18/20; A61B 6/00

(52) U.S. Cl. 606/9; 600/476

(57) ABSTRACT

There is provided a device for imaging a skin object (7) near a skin surface of a body part, comprising a light source (1) and a detector (8) for detecting radiation returning from said object (7), wherein the device further comprises an elliptical, preferably circular, polarizer (4, 19) between the source (1) and said skin (6) surface, the device comprising a 5 ratio increaser means (3, 4, 19) for increasing the ratio of radiation from said object (7) to radiation from said skin (6) surface. The ratio increaser may be an additional or the same elliptical polarizer. Using elliptically or even circularly polarized light makes hair detection independent of the orientation of hair (7) with respect to light or polarization, which renders 10 the detection more reliable. The invention also provides an imaging method and a hair-shortening device and method.

### FIELD OF THE INVENTION

The present invention relates to a device and method for imaging a skin object, such as objects on or beneath a skin surface of a body part, and to a method and device for reducing hair growth by means of said device and method.

In particular, the present invention relates in a first aspect to a device for imaging a skin object near a skin surface of a body part, comprising a source arranged to emit optical radiation having a wavelength between 600 and 2000 nm and a detector arranged to detect optical radiation returning from said object.

### BACKGROUND OF THE INVENTION

Document WO/2005/102153 dicloses a hair detection device with a source of electromagnetic radiation and an imaging sensor, and with radiation selection means. The device couples the radiation into the skin, and said radiation reaches the sensor after multiple scattering. The radiation selection means improves a ratio between said multiply scattered radiation and other, unwanted radiation. The radiation selection means may comprise complementary linear polarizers.

It was found in practice that such devices do not always provide a reliable imaging of objects, such as hairs below the skin surface. In some cases, images were very weak, with a low contrast between hairs and surrounding tissue. A reliable hair detection is not always possible in particular because of the relatively weak signals that can be extracted from below the skin surface, and because automated hair detection requires a good contrast.

### OBJECT OF THE INVENTION

It is an object of the present invention to provide a device of the kind mentioned above that renders possible a more reliable imaging of skin objects, in particular of hairs. It is also an object of the invention to provide a more reliable method of imaging skin objects.

In the present invention, a ‘skin object near a skin surface’ is an object that is present on the skin or (slightly) above or beneath the surface of the skin, such as intracutaneaous or intradermal objects, and in particular but not exclusively hairs.

### SUMMARY OF THE INVENTION

The first of the above objects is achieved with a device according to the invention, comprising a source arranged to emit optical radiation having a wavelength between 600 and 2000 nm, a detector arranged to detect optical radiation returning from said object, wherein the device further comprises an elliptical polarization means positioned in an optical path of the optical radiation between the source and said skin layer surface, the device comprising a ratio increaser means that is arranged to increase the ratio of optical radiation that is returned from said object to optical radiation that is returned from the skin layer surface, wherein said ratio increaser means is positioned in an optical path of the optical radiation between said skin layer surface and said detector.

The inventors have found that a device according to the invention that is arranged to supply elliptically polarized optical radiation provides a much more consistent detection of skin objects. The contrast between an object, such as a hair, and surrounding tissue is good. Moreover, and more importantly, the contrast is substantially independent of the orientation of the object with respect to the incoming polarization. This makes detection much more reliable and any possible subsequent operation on the detected objects more efficient.

The inventors found that the interaction of linearly polarized light with, for example, a hair depends on the orientation of the hair with respect to the polarization direction. As a result thereof, hair orientation significantly affects hair detection efficiency, which in its turn significantly decreases e.g. shaving quality, in particular during individual shaving of hairs, such as by optical means. In practice the contrast between hair and surrounding tissue may vary from hardly present to very good, which is not useful. It is assumed that the imaging becomes independent of light polarization orientation due to the fact that elliptically polarized light consists of two orthogonal, linearly polarized waves shifted in phase by 90°, such that the electric vector spirals in a helical fashion with an elliptical cross-section.

In the context of the present invention, the source may emit radiation in the indicated wavelength range, or may alternatively emit in wavelength ranges outside said range. For efficiency reasons, however, it is advantageous if the source emits substantially only in the indicated wavelength range. A substantially monochromatic source is capable of further improving efficiency and/or accuracy.

For the present application, optical radiation with the indicated wavelength will sometimes be indicated as “light” and sometimes as “optical radiation” within the indicated wavelength ranges, this is intended to mean the same thing.

The detector may be arranged to detect areawise, i.e. to image a two-dimensional area substantially simultaneously. It is, however, also possible that the detector is arranged to detect consecutively, for example in a scanning mode, such as by scanning a light beam with respect to the object or vice versa. This will be elucidated below, where also further explanations may be found concerning embodiments that are defined in the dependent claims.

In particular, the wavelength is between 800 and 1700 nm, preferably excluding the ranges 970±20 nm, 1160±20 nm, and 1440±20 nm. It was found that the indicated wavelength range and preferably outside the three indicated sub-ranges, offers a good penetration into the skin, while still achieving a good contrast with objects to be imaged, in particular hairs.

In an embodiment, the elliptical polarization means has an axial ratio of between 2:1 and 1:1, inclusive, and preferably of substantially 1:1. This means that the polarization means is able to convert light with an arbitrary polarization state, e.g. linear or unpolarized, into elliptically polarized light with the indicated axial ratio, the amplitude of the light wave in a first direction then being at the most twice the amplitude of the light wave in the perpendicular direction, and preferably substantially the same. This last particular case relates to circularly polarized light. The more the axial ratio approaches 1:1, the more constant the contrast in the detected image will be.

In a special embodiment, the elliptical polarization means positioned between the source and the skin surface constitutes a first elliptical polarization means, and the ratio increaser means comprises a second elliptical polarization means. In this embodiment, the optical radiation returning from the body part to be imaged may be sent through the second elliptical polarization means. The type and the parameters, e.g. orientation, of the second elliptical polarization means, also called elliptical polarizer herein, may be selected appropriately in order to increase the contrast between desired and undesired optical radiation.

In practice, it is important to remove from the detection signal that light that is partially reflected at the interface between the skin and a medium, such as air, water, an index-matching medium, or the like, since the reflected signal is typically much stronger than the detection signal, which prevents an efficient detection.

The light that is reflected at the medium-skin interface is reflected from a dielectric surface with a reflective index that is higher than the reflective index of a light propagation medium, which is, for example, air with n≈1 or water with n≈1.33. This introduces a 180° phase shift of one of the components of elliptically or circularly polarized light, typically the s-component. As a consequence, the elliptically polarized light that is reflected at the medium-skin interface changes the direction of polarization, from right-hand to left-hand or vice versa. Since this reflected light does not provide information useful for imaging skin objects, it is advantageous to suppress or absorb this light. This may be done with the use of e.g. a second polarization filter with a suitable polarizing action and orientation, in particular comprising an optical retardation plate.

Contrarily, part of the light that has interacted with, e.g. been scattered by an object of interest, such as a hair, does not change its polarization from left- to right-hand other vice versa. Since this is different from the polarization state of light reflected at the medium-skin interface, this distinction may be used to improve the ratio between object-interacted and skin-reflected light, e.g. by means of the ratio increaser means, such as the second elliptical polarizer.

In another embodiment, the ratio increaser means comprises the elliptical polarization means. In this case, use may be made of the polarizing properties of the first elliptical polarizer itself, as follows. As discussed above, the elliptically-polarized light reflected by the skin has a polarization state opposite to that of the incident radiation, e.g., left-hand versus right hand or vice versa. In particular, if the first elliptical polarization means produces circular polarized light from linear polarized light, the polarization state of the radiation reflected at the medium-skin interface will be converted by the first elliptical polarization means back into the linear state, but it will now be orthogonal to the original radiation. At the same time, the light that has interacted with hair will preserve its direction of rotation and will be converted by the first polarization means back into linearly polarized light, which will have the same linear polarization as initially. As a result, the linear polarization states of the radiation reflected by the skin and interacted with a hair will be orthogonal. Therefore, the reflected light can now be efficiently suppressed, e.g., by a linear polarizer. Alternatively, a polarizing beam splitter cube may be used, which will reflect and transmit different polarization components differently. The light reflected by the skin and affected by a hair can thus be spatially separated.

In particular, the elliptical polarization means comprises a linear polarizer and an optical retardation plate, such as a quarter-wave plate. This offers freedom of design for the device according to the invention. For example, these parts may be positioned at a mutual distance, with one or more other elements positioned between them, as will be elucidated below. An optical retardation plate has its usual meaning herein of a plate which is transparent to the used optical radiation and which has the property that the speed of propagation for a polarization direction in a first orientation (the “fast” axis) is higher than in the direction perpendicular thereto (the “slow” axis). This causes a phase difference between the two component parts of a light wave along those two directions. If the appropriate angle with respect to the direction of polarization of the linearly polarized light and the thickness of the retardation plate, which determines the phase difference, are suitably selected, the net result will be that the light becomes elliptically polarized. It is also possible to make circularly polarized light in a manner known to those skilled in the art.

In a special embodiment, the device comprises an optical imaging system that comprises an object optical element positioned at an object side of the optical path and a detector optical element positioned at a detector side of the optical path. This embodiment is well suited to suppress much unwanted radiation, e.g. in order to obtain information from a certain depth within the skin without having other skin layers contribute to the signal, by imaging only a well-defined portion of the body part. The object optical element may comprise, for example, a lens, and the detector optical element may comprise a pinhole or lens. Such elements are suitable to limit the field of view of the detector, for example in order to block light coming from other parts at different depths.

In particular, the device is arranged as a confocal imaging device or an optical coherence tomography device. In the case of a confocal imaging device, a focal point of a focusing lens is imaged on a confocal pinhole and transmitted through it. Only the signal generated in a focal point, i.e. at the position of the object of interest, is detected in this manner. Light originating outside of a focal plane, i.e. carrying no information about the object of interest, is focused before or after the pinhole and is rejected. This provides spatial resolution in the axial direction, i.e. in depth.

In the case of an optical coherence tomography device, spatial resolution along the axial direction is obtained by means of a low-coherence light source, for which a coherence length of approximately 30 micrometers may be selected. In OCT, a light beam from a light source is split into two parts, the so-called sample arm and the reference arm. Combining these two beams results in an interference pattern. The interference pattern only occurs if the optical paths of the sample and the reference arms coincide within the coherence length. A different depth within the sample is probed through changing of the length of the reference arm. In such devices, use may be made, and is indeed advantageously made, of one or more beam splitters, polarizing beam splitters, dichroic mirrors, or semi-transparent mirrors. Details of said arrangements are known in the art and will also be given in the description of the drawings.

Advantageously, the device according to the invention further comprises a control unit for receiving a signal from the detector. Although it may be sufficient to generate a detection signal which is subsequently processed outside the device, it is obviously advantageous if a control unit is provided to process such a signal on board. Preferably, the control unit comprises means, such as an instruction code or software, for processing such an image, or at least detection signals, in order to recognize an object in the body part that is imaged. In particular, the control unit is arranged to detect at least one hair in the body part that is imaged. Hair detection itself is known in the art, and details of such known hair detection means may be incorporated in the device of the present invention.

The invention relates, in a second aspect, to a method of imaging a skin object near a skin surface of a body part, comprising the steps of applying optical radiation to a skin layer, the optical radiation having a wavelength between 600 and 2000 nm, preferably between 800 and 1700 nm, more preferably excluding the ranges 970±20 nm, 1160±20 nm, and 1440±20 nm, and detecting optical radiation returning from the direction of said skin, wherein the applied optical radiation is elliptically polarized radiation, preferably with an axial ratio of between 2:1 and 1:1, inclusive, and more preferably of substantially 1:1. This is a method counterpart of the device of the invention and represents a use thereof. However, it is not excluded to use other devices for implementing the method of the invention. Advantageous features of the device according to the invention apply similarly to the method of the invention.

The invention relates, in a third aspect, to a method of reducing hair growth, comprising the method of imaging a skin object near a skin surface of a body part according to the second aspect of the invention, wherein the object comprises at least one hair, the method further comprising the step of supplying an amount of optical energy to at least a portion of said at least one hair that is sufficient to affect the hair's integrity. Since, according to the invention, the detection reliability is increased, the reliability and efficiency of a method of reducing hair growth will also be increased. In the context of the present invention, reducing hair growth may be achieved by severing the hair or by inflicting sufficient damage on the hair for it to be shed, or by stimulating dormancy of the hair-generating tissue, etc. These methods comprise epilation and shaving, for example.

In the method according to the second aspect, a device according to the first aspect is preferably used.

The method may advantageously comprise the steps of scanning a body part, advantageously a surface or subsurface area thereof, processing the image signal(s), and supplying an amount of energy to one or more detected hairs that is sufficient to reduce the growth thereof. This may be done, for example, by first imaging the body part as a whole, processing, and supplying energy. The steps may be performed substantially simultaneously. For example, a linear scan is made, a maximum brightness signal is detected, and one or more energy pulses are supplied to a location corresponding to that maximum. Note that it may happen that a hair image provides first a bright signal at a hair-skin interface, followed by a relatively low intensity signal from the cortex, followed by a very bright signal from the medulla, and symmetrically back. This gives three peaks, with the strongest in the middle. In other cases a hair may appear as a homogeneous bright object. In such a case, supplying energy could be optimized accordingly. Other rules and methods may also be used.

The invention relates, in a fourth aspect, to a device according to the first aspect of the invention, additionally comprising a hair growth reducing means that is arranged to supply an amount of optical energy to at least a portion of said at least one hair that is sufficient to affect the hair's integrity. The hair growth reducing means is operatively coupled to the imaging device, in particular to achieve a good correlation between the position of a detected skin object and a positioning of the hair growth reducing means.

Any hair growth reducing means may be used both in the method and in the device for reducing growth of hairs. In particular, a hair growth reducing means that affects hair subcutaneously is comprised in the device or method for reducing growth of hairs according to the invention. In particular, a hair growth reducing means comprises a means for providing sufficient optical energy to damage or cut the hair.

In a special embodiment, the source comprises a laser, the device preferably further comprising a laser power control means that is arranged to switch an emitted laser power between two different non-zero values. A first value is e.g. a low value for imaging, and a second value is e.g. a high value for cutting hairs. A laser is a very suitable source of optical radiation since it is able to provide a high power density in a very small focal area, which is useful because there are high losses due to absorption and suppression of unwanted light. In various circumstances, focusing onto a small focal spot, roughly at most of the order of the diameter of the object to be imaged, is advantageous. In the case of hairs, a diameter of a few tens of micrometers is useful, although other diameters are not excluded. Such a diameter is easily achieved with a laser and a focusing lens. Other sources to be used in hair imaging are not excluded, however, for example LEDs may also be useful, because they too can provide more or less monochromatic light in useful wavelength areas, and with relatively high power. However, focusing below about 0.1 mm is not efficiently achieved. This may make the use of, for example, the pinhole advantageous.

A general remark to be made here is that objects to be imaged relate in particular to hairs. Hairs have the property of interacting with polarized light in a way that depends on the orientation of the hair with respect to the polarization vector. There are other structures in body parts that show such properties, such as collagen fiber bundles. These, however, are much smaller, and the effect is much less pronounced.

### BRIEF DESCRIPTION OF THE DRAWINGS

The invention will now be elucidated with reference to the drawings, which show a number of non-limiting embodiments, and in which:

FIG. 1 shows a highly diagrammatic embodiment of a device according to the invention;

FIG. 2 diagrammatically shows an embodiment of a hair-shortening device according to the invention;

FIG. 3 diagrammatically shows a further embodiment of a hair-shortening device according to the invention; and

FIG. 4 diagrammatically shows a variation of the further embodiment of FIG. 3.

### DETAILED DESCRIPTION OF EXAMPLES

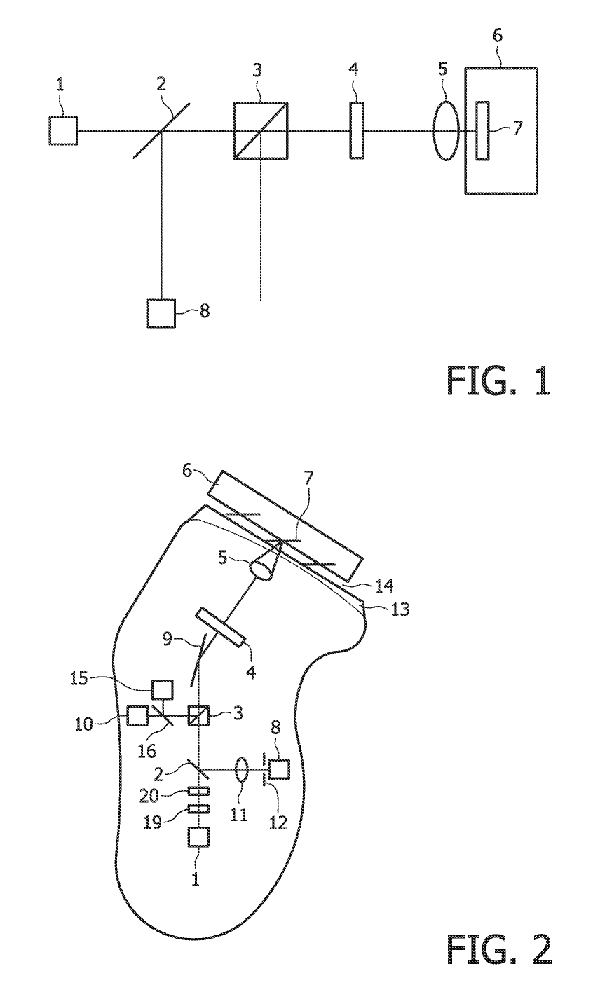

FIG. 1 shows a highly diagrammatic embodiment of the device according to invention. Herein, 1 is a light source for imaging, 2 denotes a beam splitter, 3 denotes a polarizing beam splitter, 4 denotes a quarter-wave plate, 5 denotes an imaging lens, 6 denotes tissue, 7 denotes a hair, and 8 denotes a detector.

The light source 1, such as a LED, but preferably a laser, emitting near infrared radiation of a wavelength of e.g. 834 or 1310 nm, emits with a linear polarization, e.g. in a p-state. If necessary, a linear polarizer may be added. The beam passes the beam splitter 2 and the polarizing beam splitter 3. Next, the beam passes a quarter-wave plate 4 and becomes circularly polarized, at least if the electric field factor of the beam is oriented at an angle of 45° to the fast axis of the plate 4. Next, the beam passes the imaging lens 5 and enters the tissue 6, such as skin. On entering, a major portion of the radiation will be reflected. The portion that enters the tissue 6 will be partly returned from hair 7 through scattering, reflection, or by some other mechanism.

The returning radiation will again go through imaging lens 5 and through quarter-wave plate 4. Then, there will be a difference between light reflected from the medium-skin interface and light returning from the hair 7. Light reflected at the medium-skin interface will change the direction of circular polarization, such as from left-hand to right-hand. When this light propagates through the plate 4, the resulting polarization state will be orthogonal to that of the incoming state, in this case s instead of p. This light will not pass through the polarizing beam splitter 3 but will be reflected and thus removed from the beam that travels to the detector 8.

Contrarily, at least part of the light scattered by the hair will preserve its direction of rotation and will thus be converted by the first polarization means back into the linear polarization, which will be the same as the initial polarization, in this case the p-state. The polarizing beam splitter 3 will transmit this light, which then travels to the beam splitter 2, which will reflect part of the light to the detector 8. Summarizingly, the light reflected at the medium-skin interface will be suppressed, whereas light returning from the hair 7 will be transmitted towards the detector 8, and their ratio will be improved, i.e. decreased. The hair detection efficiency is found to be independent of hair orientation.

FIG. 2 diagrammatically shows an embodiment of a hair-shortening device. Herein, as in all of the drawings, similar parts are denoted by the same reference numerals. The device furthermore comprises a mirror 9 for adjusting the light path, a second photodetector 10, a detector lens 11, and a pinhole 12 in front of the “first” detector 8. Furthermore, there is provided a contact window 13, while 14 denotes immersion fluid.

15 denotes a light source for severing hairs, 16 is a second beam splitter. 19 denotes a linear polarizer and 20 denotes a half-wave plate.

The present device, in the form of an ordinary shaver, comprises the parts already mentioned in the description of FIG. 1 and many more, the function of which will be explained below.

Radiation emitted by the light source 1 passes through a linear polarizer 19 to provide linearly polarized light in case the light source 1 would emit substantially non-polarized light. In practice, light sources such as superluminescent diodes (SLD) or a superconductor optical amplifier (SOH) may be used, which have a short coherent length and are capable of emitting elliptically polarized light with a very high axial ratio, e.g. of about 20:1, which almost represent linearly polarized light. The half-wave plate 20 may be used to rotate the linear polarization state to a preferred orientation.

Again, the beam will travel through the beam splitter 2 and the polarizing beam splitter 3 and will then hit the mirror 9 for adjusting the beam. This beam may be used for scanning the surface of the tissue 6, e.g. in two dimensions.

The reflected radiation will be split by the polarizing beam splitter 3 into radiation “mainly” reflected from the skin and radiation returned from a hair 7. The skin-reflected radiation will travel towards second detector 10, which may be a photodiode or other photodetector. If this detector receives a signal, this may indicate that an object, of course preferably skin, is present in front of the device. In other words, this second detector 10 may serve as a proximity detector.

The radiation returned from a hair 7 will travel via beam splitter 2 and detector lens 11 trough pinhole 12 to the detector 8. The detector lens 11 and the pinhole 12 serve to image a focal point of the imaging lens 5 onto the detector in order to increase the ratio of desired radiation containing information on the presence of hairs to radiation from neighboring parts that do not contain any useful information.

Not shown is a control unit for processing the signals from the detector 8. Such a control unit, which may also control the various light sources, mirrors, etc., may be embodied as a suitable IC or the like. This control unit may also comprise image processing software, e.g. for hair recognition.

The light source 15 for cutting hair is shown as a separate light source, such as a second laser with a relatively higher power. Alternatively, source 15 and source 1 may be one and the same source, but with a switchable power. The radiation from the light source 15 for cutting hairs is projected into the beam via an additional beam splitter 16. If necessary, a linear polarizer may be provided. If the proximity sensor 10 is not used, the light source 15 may also be coupled into the beam directly, i.e. without a second beam splitter 16.

The optical or contact window 13 and the immersion fluid 14, which are optional, may serve to improve the penetration properties of the radiation into the skin. For example, the fluid 14 may be an index matching fluid, having an index of refraction which is halfway between that of the optical window and that of the skin. Preferably, all refractive indices are substantially equal. This also lowers the reflection from the skin. The fluid may also be selected for the purpose of cooling the skin, or treating it otherwise. Furthermore, although the contact window 13 is optional, it helps in serving as a reference for determining positions of skin objects, such as the hairs 7.

The embodiment shown here is an example of a device that uses confocal detection to reject out-of-focus light and to obtain depth-resolved information.

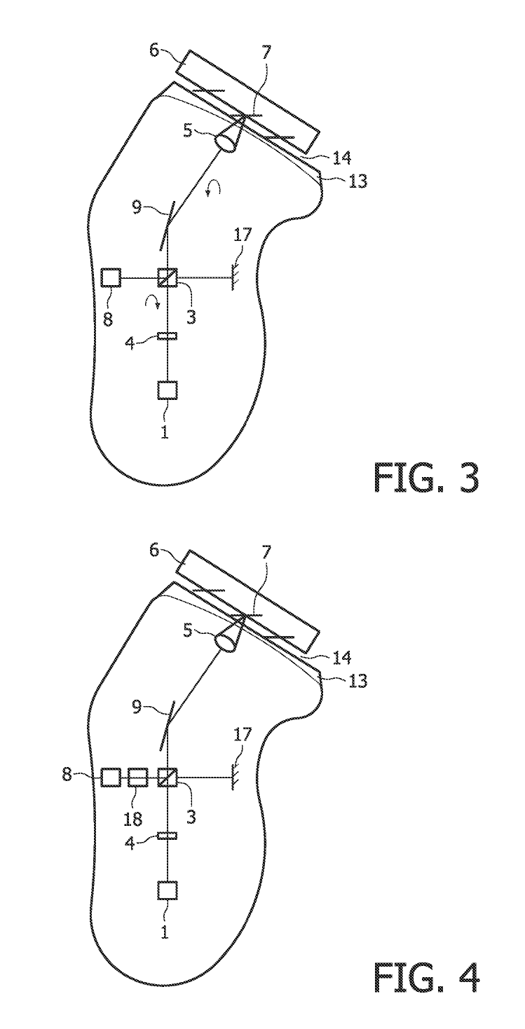

FIG. 3 diagrammatically shows an embodiment of the device that uses time domain optical coherence tomography for hair detection.

The device comprises a light source 1, a quarter-wave plate 4, a polarizing beam splitter 3, a detector 8, a mirror for adjusting the optical path 9, an imaging lens 5, and a movable mirror 17.

The light source 1 may be a super luminescent diode, a superconductor optical amplifier, or any other light source that is suitable for optical coherence tomography (OCT).

The radiation emitted will travel partly via movable mirror 17 and partly via the path through the imaging lens 5 and the skin 6/hair 7. The optical path lengths are selected so as to be substantially equal, which will cause interference effects at the detector 8. This works in much the same way as a Michelson-Morley interferometer, and those skilled in the art will know the details. The mirror 17 will be movable to allow correction of the changing path length when moving the mirror 9 for adjusting or when selecting a different depth in the skin. Note that, for any received information to be depth-dependent, the coherence length of the light source 1 should be comparable to the desired resolution.

This embodiment, too, is able to provide position-dependent information on whether or not a hair (or other object) is present in the tissue (e.g. the skin) 6.

Not shown separately is a device for shortening hair, or at least sufficiently damaging the hair to be shortened. This device may be the same as the light source 1, which would then have a controllable power level.

Note that the movable mirror 17 may be replaced by any other implementation of a variable time delay line for varying the optical path length of the reference arm of the OCT.

FIG. 4 diagrammatically shows a variation of the embodiment of FIG. 3, in particular a Fourier domain OCT for hair detection. Most of the parts of this embodiment are the same as in the time domain OCT embodiment of FIG. 3. However, a spectrograph 18 is additionally shown, comprising a diffraction element, such as a diffraction grating. This embodiment, also called spatially encoded frequency domain OCT, can give information on various depths in a single exposure. Further details of this technique may be found, for example, in the Handbook of optical coherence tomography, Ed. B. E. Bouma, G. J. Tearney, Marcel Dekker, Inc. New York, 2002. Other embodiments are not excluded.

1. A device for imaging a skin object (7) near a skin surface of a body part, comprising:
a source (1) arranged to emit optical radiation having a wavelength between 600 and 2000 nm;
a detector (8) arranged to detect optical radiation returning from said object (7);
wherein the device further comprises an elliptical polarization means (4, 19) positioned in an optical path of the optical radiation between the source (1) and said skin (6) surface, the device comprising a ratio increaser means (3, 4, 19) that is arranged to increase the ratio of optical radiation that returns from said object (7) to optical radiation that returns from said skin (6) surface,
said ratio increaser means (3, 4, 19) being positioned in an optical path of the optical radiation between said skin (6) surface and said detector (8).

2. The device according to claim 1, wherein the wavelength is between 800 and 1700 nm, preferably excluding the ranges of 970±20 nm, 1440±20 nm, and 1160±20 nm.

3. The device according to claim 1, wherein the elliptical polarization means (4, 19) has an axial ratio of between 2:1 and 1:1, inclusive, and preferably of substantially 1:1.

4. The device according to claim 1, wherein the elliptical polarization means constitutes a first elliptical polarization means, and wherein the ratio increaser means (3, 4, 19) comprises a second elliptical polarization means.

5. The device according to claim 1, wherein the ratio increaser means comprises the elliptical polarization means (4, 19).

6. The device according to claim 1, wherein the elliptical polarization means comprises a linear polarizer (19) and an optical retardation plate (4).

7. The device according to claim 1, comprising an optical imaging system that comprises an object optical element (5) positioned at an object side of the optical path and a detector optical element (8, 10) positioned at a detector side of the optical path.

8. The device according to claim 1, arranged as a confocal imaging device or an optical coherence tomography device.

9. The device according to claim 1, further comprising a control unit for receiving a signal from the detector (8, 10) and for processing said signal into an image of said body part.

10. A method of imaging a skin object (7) near a skin (6) surface of a body part, comprising the steps of:
applying optical radiation to a skin (6) layer, the optical radiation having a wavelength between 600 and 2000 nm, preferably between 800 and 1400 nm, more preferably excluding the ranges of 970±20 nm and 1200±20 nm;
detecting optical radiation returning from the direction of said skin (6) layer;
wherein the applied optical radiation is elliptically polarized radiation, preferably with an axial ratio of between 2:1 and 1:1, inclusive, and more preferably of substantially 1:1.

11. The method of claim 10, wherein the detecting step is carried out substantially confocally or in accordance with an optical coherence tomography technique.

12. A method of reducing hair growth, comprising the method of imaging a skin object near a skin (6) surface of a body part according to claim 10, wherein the object comprises at least one hair (7), the method further comprising the step of supplying an amount of optical energy to at least a portion of said at least one hair (7) that is sufficient to affect the hair's integrity.

13. A device for reducing growth of hairs, comprising a device according to claim 1, comprising a hair growth reducing means that is arranged to supply an amount of optical energy to at least a portion of said at least one hair (7) that is sufficient to affect the hair's integrity.

14. The device according to claim 13, wherein the source (1) comprises a laser (1, 15), the device preferably further comprising a laser power control means that is arranged to switch an emitted laser power between two different non-zero values.
